(19) United States
(12) Patent Application Publication
(10) Pub. No.: US 2023/0020665 A1
(43) Pub. Date: Jan. 19, 2023

(54) APPARATUS, SYSTEMS, AND METHODS OF TRANSFERRING LIQUIDS CONTAINING AGGREGATES

(71) Applicant: Siemens Healthcare Diagnostics Inc., Tarrytown, NY (US)

(72) Inventors: Chung-Hsuan Huang, Newark, DE (US); William D. Dunfee, Newark, DE (US)

(73) Assignee: Siemens Healthcare Diagnostics Inc., Tarrytown, NY (US)

(21) Appl. No.: 17/756,254

(22) PCT Filed: Nov. 18, 2020
(86) PCT No.: PCT/US2020/061123

§ 371 Date: May 19, 2022

Related U.S. Application Data
(60) Provisional application No. 62/939,494, filed on Nov. 22, 2019.

(51) Int. Cl. G01N 35/00; G01N 33/543

(52) U.S. Cl. CPC G01N 35/0098; G01N 33/54326; G01N 2035/00564

(57) ABSTRACT

An apparatus configured to receive particles in a liquid includes: a housing comprising a housing inlet and a housing outlet; and a mesh located in the housing between the housing inlet and the housing outlet, the mesh having spaces greater than a greatest transverse dimension of the particles. The apparatus operates to break up agglomerates of the particles, such as agglomerates of magnetic particles. Other systems and methods of receiving and transferring liquids containing particles having a propensity to agglomerate are disclosed, as are other aspects.

### CROSS REFERENCE TO RELATED APPLICATION

This application claims the benefit of U.S. Provisional Patent Application No. 62/939,494, entitled “APPARATUS, SYSTEMS, AND METHODS OF TRANSFERRING LIQUIDS CONTAINING AGGREGATES” filed Nov. 22, 2019, the disclosure of which is hereby incorporated by reference in its entirety for all purposes.

### FIELD

The present disclosure relates to apparatus, systems, and methods of transferring liquids containing aggregates.

### BACKGROUND

In analytical testing, one or more liquids may be pumped from one location to another using one or more pumps. For example, liquids may be pumped to a waste collection container and/or from a waste collection container within an analytical test instrument. Some reactions performed by the analytical test instrument can use magnetic particles dispersed within liquids. In some embodiments, the magnetic particles may have a transverse dimension (e.g., diameter) in a range from 10 μm to 100 μm. The pumps may be diaphragm pumps, for example, that include valves made of flexible materials.

### SUMMARY

According to a first aspect, an apparatus configured to receive particles in a liquid is disclosed. The apparatus includes a housing comprising a housing inlet and a housing outlet; and a mesh located in the housing between the housing inlet and the housing outlet, the mesh having spaces greater than a greatest transverse dimension of the particles.

According to a second aspect, a clinical diagnostic analyzer is disclosed. The system includes a pump configured to pump a liquid containing particles; a mesh apparatus configured to disassociate aggregates of the particles, the mesh apparatus including a housing comprising a housing inlet and a housing outlet coupled to the pump; and a mesh located in the housing between the housing inlet and the housing outlet, the mesh having spaces greater than a greatest transverse dimension of the particles.

In a method aspect, a method of transferring a liquid containing particles is disclosed. The method includes providing a mesh having spaces, the spaces having widths greater than a greatest transverse dimension the particles; and moving the liquid containing the particles through the mesh, wherein the moving disassociates aggregates of the particles.

Still other aspects, features, and advantages of the present disclosure may be readily apparent from the following description by illustrating a number of example embodiments and implementations. The present disclosure may also be capable of other and different embodiments, and its several details may be modified in various respects, all without departing from the scope thereof. Accordingly, the drawings and descriptions are to be regarded as illustrative in nature, and not as restrictive. The disclosure is to cover all modifications, equivalents, and alternatives falling within the scope of the claims.

### BRIEF DESCRIPTION OF THE DRAWINGS

The drawings, described below, are for illustrative purposes only and are not necessarily drawn to scale. The drawings are not intended to limit the scope of the disclosure in any way. Like elements throughout are identified using like numerals.

FIG. 1A illustrates a block diagram of a liquid transfer system including a mesh apparatus according to one or more embodiments of the disclosure.

FIG. 1B illustrates a block diagram of a liquid transfer system including a cross-sectioned view of a mesh apparatus in the form of a container according to one or more embodiments of the disclosure.

FIG. 2A illustrates a partial cross-sectioned view of a pump according to one or more embodiments of the disclosure.

FIG. 2B illustrates a cross-sectioned view of an inlet valve of a pump, wherein the inlet valve is in an open state and is passing small clusters of magnetic particles according to one or more embodiments of the disclosure.

FIG. 2C illustrates a side cross-sectioned view of a valve of a pump, wherein the valve is in an open state and is being damaged by large aggregates of magnetic particles.

FIG. 3A illustrates a front elevation view of a mesh with various magnetic particles and aggregates of magnetic particles passing through the mesh according to one or more embodiments of the disclosure.

FIG. 3B illustrates a side elevation view of a mesh with various magnetic particles and aggregates of magnetic particles preparing to pass through the mesh according to one or more embodiments of the disclosure.

FIG. 3C illustrates a side elevation view of a mesh after various magnetic particles and small aggregates of magnetic particles have passed through the mesh according to one or more embodiments of the disclosure.

FIG. 4A illustrates a plan view of a housing first portion of a mesh apparatus without a mesh located therein according to one or more embodiments of the disclosure.

FIG. 4B illustrates a plan view of a housing first portion of a mesh apparatus with a circular mesh located therein according to one or more embodiments of the disclosure.

FIG. 4C illustrates a side elevation view of a mesh apparatus according to one or more embodiments of the disclosure.

FIG. 5A illustrates a plan view of a housing first portion of a mesh apparatus including tabs extending from the housing first portion according to one or more embodiments of the disclosure.

FIG. 5B illustrates a plan view of housing first portion of a mesh apparatus including tabs extending from the housing first portion and a circular mesh located in the housing first portion according to one or more embodiments of the disclosure.

FIG. 5C illustrates a side elevation view of a mesh apparatus according to one or more embodiments of the disclosure.

FIG. 6 illustrates a flowchart of a method of transferring a liquid containing magnetic particles according to one or more embodiments of the disclosure.

### DETAILED DESCRIPTION

As discussed above, the liquids containing magnetic particles (sometimes referred to as “magnetic beads”) may be pumped to one or more locations (e.g., to a waste collection container) using one or more pumps after testing is completed. The magnetic particles may be made from ferromagnetic materials. The magnetic particles include particles that respond to a magnetic field, such as by movement thereof. In some embodiments, the ferromagnetic materials may be polymer-based and in other embodiments, the ferromagnetic materials may be metal-based. The magnetic particles may include an organic or inorganic coating. In addition, the magnetic particles may not dissolve in the liquids. Over time and in transit, the magnetic particles may be attracted to one another and form aggregates of magnetic particles having transverse dimensions that are much greater than the transverse dimensions of the individual magnetic particles. For example, some of the magnetic particle aggregates may have transverse dimensions of 1.3 mm or greater.

The pumps used in such systems may be diaphragm pumps that include a diaphragm that may oscillate. The oscillating diaphragm may move the liquid containing the magnetic particles through an inlet valve of the pump and into a pump chamber. The oscillating diaphragm may then move the liquid and particles from the pump chamber, through an outlet valve of the pump, and to an outlet of the pump. The amount of liquid being moved may be small, so the pump's inlet valve and outlet valve may also be quite small. In some embodiments the inlet valve and/or the outlet valve may have a transverse dimension (e.g., diameter) of about 1.3 mm, for example. In some embodiments, the inlet and outlet valves may include flexible flaps that open and close and operate as check valves in order to control reverse flow of the liquid and magnetic particles.

Flow of the aggregates of magnetic particles through the valves may damage and/or clog the pumps. For example, larger aggregates may impinge on or become caught in the valves and may damage the valves such as by prematurely wearing the flexible flaps. In some instances, larger aggregates may prevent the flexible flaps from closing correctly, which prevents the pump from transferring liquids efficiently. In these instances, the pumps may be damaged and may have to be prematurely serviced and/or replaced. Furthermore, these instances can disable the analytical test instruments including the pump causing unwanted down time.

The above-described problems caused by aggregates of magnetic particles can be alleviated by the apparatus, systems, and methods disclosed herein. In some embodiments, a mesh apparatus including a mesh is coupled to a liquid line that transfers liquid containing magnetic aggregates. The mesh may have spaces (e.g., openings) that are larger than the largest transverse dimension of the magnetic particles, which prevents the mesh from functioning as a filter. Accordingly, all the individual magnetic particles may pass through the mesh. The aggregates acquire energy as they move in the liquid line. Aggregates of magnetic particles are disassociated (e.g., broken apart) into individual magnetic particles or smaller aggregates when the aggregates collide with the mesh. For example, the energy expended by the aggregates contacting the mesh is greater than forces holding the aggregates together, so the aggregates break apart (i.e., they disassociate) and pass through the mesh. In some embodiments, magnetic forces may hold the aggregates together. In some embodiments, adhesion forces may hold the aggregates together. For example, the magnetic particles may be coated with proteins or other chemicals that cause the magnetic particles to adhere to one another and form the aggregates. In some embodiments, both adhesion forces and magnetic forces may hold the aggregates together.

In some embodiments, a dimension of a largest space of the mesh is smaller than a transverse dimension of an inlet valve of the pump. Accordingly, large aggregates are broken apart by the mesh, so only aggregates having sizes less than or equal to the largest space of the mesh can pass through the mesh and be received in the inlet valve of the pump. These aggregates are smaller than the transverse dimension of the inlet valve, so the aggregates pass through the inlet valve without clogging or appreciably damaging the inlet valve.

In some embodiments, the dimension of the spaces in the mesh are about 1.2 mm and the transverse dimension of the inlet valve is about 1.3 mm. The mesh may be any suitable structure with multiple openings (spaces formed therein). For example, the mesh may be a wire mesh made of stainless steel wires that are woven to form the spaces. The spaces are the openings through which the magnetic particles or smaller aggregates can pass. The wires may have diameters of about 0.254 mm and the mesh may have an open area of from 31% to 41% (nominally about 36%), for example. The open area is the area through which flow can pass, such as through a center plane of the mesh. The mesh may be made of other materials, such as other nonmagnetic materials and may have other suitable dimensions smaller than the largest aggregates. The mesh may have other suitable dimensions smaller than the inlet valve.

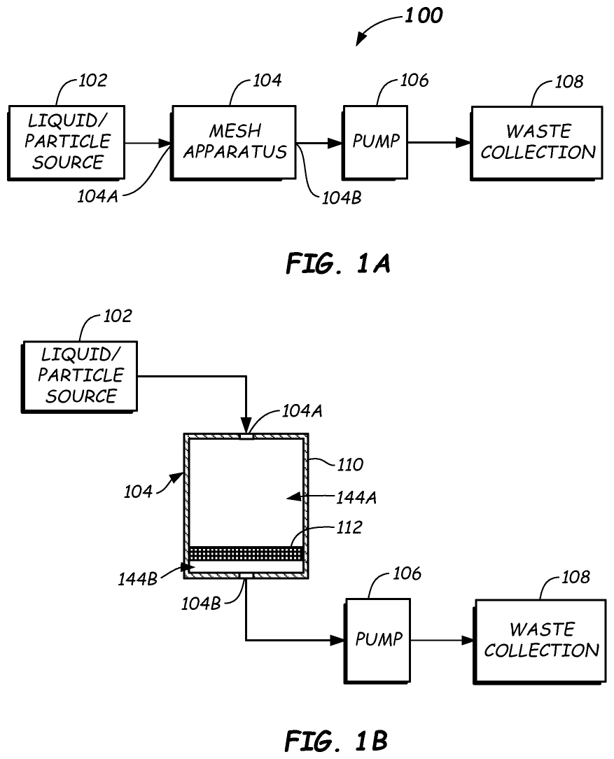

The above-described embodiments, along with other apparatus, systems, and methods are further described in greater detail below with reference to FIGS. 1A-6.

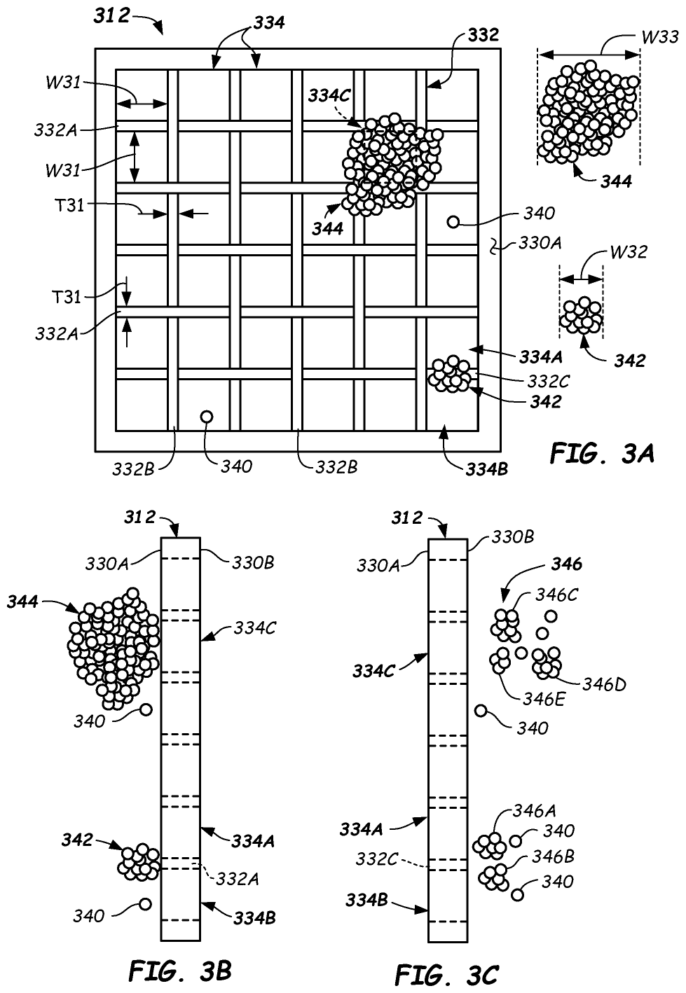

Reference is made to FIG. 1A, which illustrates a block diagram of a liquid transfer system 100 that may be configured to transfer liquids containing particles, such as magnetic particles, between different locations. The liquid transfer system 100 may transport liquids that have magnetic particles (e.g., like magnetic particle 340—FIG. 3B) suspended therein. The magnetic particles may have transverse dimensions (diameters) in a range from 10 μm to 100 μm. In some embodiments, the magnetic particles may have other transverse dimensions. The magnetic particles may be coated with a bonding agent such as silicon, and may be used as analyte bonding agents for immunoassays or other chemical/diagnostic analysis. Thus, the liquid transfer system 100 may be implemented within an immunoassay instrument, clinical diagnostic analyzer, or the like.

The liquid transfer system 100 may include or be coupled to a liquid/particle source 102. The liquid/particle source 102 may be any source of liquid that contains particles that have a propensity to agglomerate, such as magnetic particles. In some embodiments, the liquid/particle source 102 may be a cuvette, a well, or other vessel where a liquid containing magnetic particles was contained. In other embodiments, the liquid/particle source 102 may be a primary waste collection container (not shown) that is configured to accumulate waste liquids containing magnetic particles discarded after processing.

The liquid/particle source 102 may be coupled to an inlet 104A of a mesh apparatus 104, which is described in greater detail below. The mesh apparatus 104 functions to break up (i.e., disassociate) aggregates of the particles (e.g., magnetic particles) into individual particles (e.g., individual magnetic particles) and/or smaller aggregates.

A pump 106 may be coupled to an outlet 104B of the mesh apparatus 104 and may be configured to pump liquids containing magnetic particles and smaller aggregates. The pump 106 may control the flowrate of the liquid through the mesh apparatus 104. The flowrate is one parameter that controls the velocity of the magnetic particles through the mesh apparatus 104, which provides energy to the magnetic aggregates so that the magnetic aggregates may break apart or disassociate when colliding with the mesh apparatus 104. In some embodiments, the flowrate is about 0.3 L/min through the mesh apparatus 104. In some embodiments, the flowrate may be in a range from 0.2 L/min to 0.4 L/min. The pump 106 may provide other flowrates through the mesh apparatus 104.

As described in greater detail below, the pump 106 may be a diaphragm pump that includes valves made of flexible material. The mesh apparatus 104 breaks the aggregates of magnetic particles into small aggregates or individual magnetic particles of sufficiently small transverse dimension so the aggregates do not damage and/or clog the pump 106. For example, the smaller aggregates and individual magnetic particles may not clog and/or damage the flexible valves. The pump may discharge the liquid containing the magnetic particles and/or small aggregates to a waste collection 108.

Reference is now made to FIG. 1B, which illustrates a block diagram of an embodiment of the liquid transfer system 100 including a cross-sectioned view of an embodiment of the mesh apparatus 104 in the form of a container 110. The container 110 may store liquid containing magnetic particles until such a time that the pump 106 may remove the liquid from the container 110. The container 110 may include a mesh 112 located between the inlet 104A and the outlet 104B. The mesh 112 may be located and configured within the container 110 so that all the liquid flowing between the inlet 104A and the outlet 104B passes through the mesh 112. Thus, all the magnetic particles and aggregates of magnetic particles in the liquid pass through the mesh 112 and are disassociated as described herein.

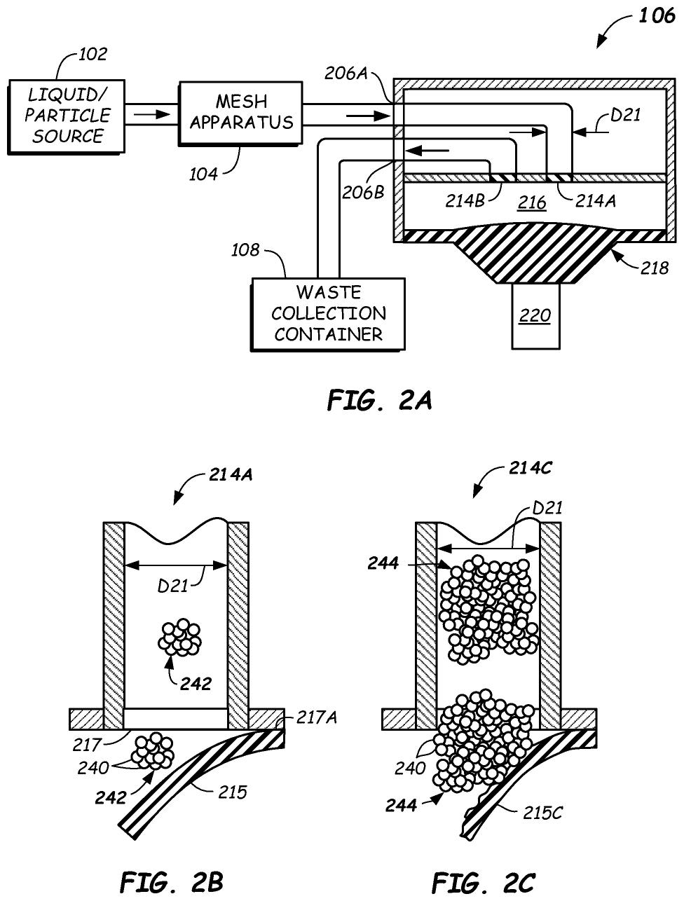

Additional reference is made to FIGS. 2A and 2B. FIG. 2A illustrates a partial cross-sectioned view of the pump 106 (e.g., a diaphragm pump) that may be similar or identical to the pump 106 (FIGS. 1A-1B). FIG. 2B illustrates a cross-sectioned side view of an inlet valve 214A in an open state and passing small aggregates of magnetic particles. The pump 106 may include an inlet 206A coupled to the mesh apparatus 104. The pump 106 may also include an outlet 206B that is coupled to the waste collection 108 or other destination. The pump 106 may include an inlet valve 214A coupled to the inlet 206A and an outlet valve 214B coupled to the outlet 206B.

FIG. 2B illustrates an enlarged view of the inlet valve 214A, which may be substantially similar to the outlet valve 214B. The inlet valve 214A may include a flap 215 that seals against a sealing surface 217 to close the inlet valve 214A and is unsealed from the sealing surface 217 to open the inlet valve 214A. The flap 215 may attach to the sealing surface 217 at a location 217A. The flap 215 may be made of a flexible material such as ethylene propylene diene monomer (EPDM), perfluoroelastomer (FFKM), or other suitable polymer, for example. The inlet valve 214A may have a transverse inlet dimension (e.g., diameter) D21, which may be about 1.3 mm, for example. The inlet valve 214A may have other transverse dimensions.

The pump 106 may also include chamber 216, a diaphragm 218, and an actuator 220 coupled to the diaphragm 218. A motor (not shown) may be coupled to the actuator 220 in a manner that provides for movement of the diaphragm 218. In use, the diaphragm 218 is pulled down by the actuator 220, which pulls liquid containing magnetic particles through the inlet valve 214A and into the chamber 216. The diaphragm 218 is then pushed up by the actuator 220, which pushes liquid from the chamber 216, though the outlet valve 214B, and out the outlet 206B.

As described above, should large aggregates of magnetic particles enter the inlet valve 214A, the aggregates may clog and/or damage the inlet valve 214A. As shown in FIG. 2B, small aggregates 242 of magnetic particles 240 are small enough to pass though the inlet valve 214A without clogging or damaging the flap 215. For example, the small aggregates 242 have passed through the mesh apparatus 104 and may be much smaller than the transverse inlet dimension D21 because they have been broken apart from large aggregates by the mesh apparatus 104.

FIG. 2C illustrates a side cross-sectioned view of a valve 214C in an open state and being damaged by large aggregates 244 of magnetic particles 240 from a conventional system not including the mesh apparatus 104. The valve 214C is not preceded by a mesh apparatus 104 as described herein, so large aggregates 244 of magnetic particles 240 have entered the valve 214C. For example, in some embodiments, the large aggregates 244 may have transverse dimension close to the transverse inlet dimension D21 and may clog and/or damage the valve 214C. As shown in FIG. 2C, the large aggregates 244 have damaged the flap 215C, which prevents the valve 214C from closing properly. In some embodiments, the large aggregates 244 may erode parts of the valve 214C to the point where portions of the valve 214C are removed, which disrupts proper valve sealing. In some embodiments, the large aggregates 244 have sizes within 1.0 mm of the transverse inlet dimension D21.

Additional reference is made to FIGS. 3A-3C, which illustrate different views of an embodiment of a mesh 312 included within a mesh apparatus. The mesh 312 may be similar or identical to the mesh 112 (FIG. 1B). FIG. 3A illustrates a front elevation view of the mesh 312 with various magnetic particles and large aggregates 342, 344 of magnetic particles preparing to pass through the mesh 312. FIG. 3B illustrates a side view of the mesh 312 of FIG. 3A with various magnetic particles and aggregates of magnetic particles preparing to pass through the mesh 312. FIG. 3C illustrates a side view of the mesh 312 after magnetic particles 340 and small aggregates 346 of magnetic particles have passed through the mesh 312. The small aggregates 346 may have been broken from large aggregates 342, 344 that collided with the mesh 312.

The mesh 312 may include a first side 330A and a second side 330B that is opposite the first side 330A. The first side 330A may be referred to as an inlet and the second side 330B may be referred to as an outlet. The mesh 312 may include a plurality of members 332 that intersect or overlap in a weave to form a plurality of spaces 334 (e.g., openings) that extend between the first side 330A and the second side 330B. The mesh 312, including the members 332, may be made of nonmagnetic materials so the magnetic particles are not attracted to the mesh 312.

The members 332 may include one or more first members 332A extending in a first direction and one or more second members 332B extending in a second direction, such as perpendicular to the first direction, as shown. In the embodiment depicted in FIG. 3A, the first members 332A are shown extending in a horizontal direction and the second members 332B are shown extending in a vertical direction. In some embodiments, the mesh 312 may be made from a single piece of material wherein the spaces 334 are formed (e.g., cut) into the single piece of material. In other embodiments, the mesh 312 may be made from woven materials. For example, the first members 332A may be woven with the second members 332B to form the spaces 334. In some embodiments, the first members 332A are first wires extending in a first direction and the second members 332B are second wires extending in a second direction wherein the first wires are woven with the second wires.

The spaces 334 may be square in plan view and may have widths W31. The spaces 334 may have other shapes, such as circular or rectangular. The members 332 may have thicknesses T31 (or diameters equal to T31). For example, the widths W31 may be the same distances between the first members 332A and the second members 332B. In addition, the first members 332A and the second members 332B may have the same thicknesses T31. In some embodiments the widths W31 are less than the transverse inlet dimension D21 (FIG. 2) of the inlet valve 214A (FIG. 2) of the pump 106. For example, the widths W31 may be at least between 0.5 mm and 1.5 mm smaller than the transverse inlet dimension D21. In some embodiments, the widths W31 are 1.0 mm smaller than the transverse inlet dimension D21. By having the widths W31 smaller than the transverse inlet dimension D21, aggregates of magnetic particles as large as or larger than the transverse inlet dimension D21 are prevented from entering the inlet valve 214A and clogging and/or damaging the inlet valve 214A. In some embodiments, the transverse inlet dimension D21 is 1.3 mm and the widths W31 are 1.2 mm. In some embodiments, the mesh 312 is sized to break up large aggregates 342, 344 into small aggregates 346, wherein the small aggregates 346 have maximum transverse dimensions 95% or less than the transverse inlet dimension D21.

In some embodiments, the first members 332A and the second members 332B are made of wires, such as stainless steel T-316 wire. The first members 332A and the second members 332B may be made of other materials, such as other nonmagnetic materials. In some embodiments, the thickness T31 is the thickness (diameter) of the wires and is in a range from 0.127 mm to 0.381 mm. In some embodiments, the thickness T31 is about 0.254 mm. The open area, which is the percentage of a surface area of the mesh 312 that is made up of spaces 334, may be in a range from 31% to 41%. In some embodiments, the open area is 36%.

FIGS. 3A-3B illustrate magnetic particles 340 and large aggregates 342, 344 of magnetic particles interacting with the mesh 312. The interaction breaks the large aggregates 342, 344 into individual magnetic particles 340 and small aggregates 346. The magnetic particles 340 and large aggregates 342, 344 and small aggregates 346 of magnetic particles may not be drawn to scale relative the members 332 and the spaces 334. As shown, magnetic particles 340 may pass through the spaces 334. In some embodiments, the magnetic particles 340 may have a transverse dimensions (e.g., diameters) in a range from 10 μm to 100 μm, which is smaller than the width W31 of the spaces 334.

A first large aggregate 342 may be formed from a plurality of magnetic particles 340. The first large aggregate 342 is shown in FIGS. 3A and 3B colliding with a member 332C located between a space 334A and a space 334B. The member 332C may be one of the first members 332A. The first large aggregate 342 may be traveling at a velocity approximately equal to the velocity of the liquid passing through the mesh 312. Accordingly, the first large aggregate 342 has momentum and energy. When the first large aggregate 342 collides with the member 332C, the energy of the collision breaks or disassociates the first large aggregate 342 into individual magnetic particles 340 and/or small aggregates 346. For example, the energy expended in the collision is greater than the magnetic force holding the first large aggregate 342 together. As shown in FIG. 3A, the first large aggregate 342 originally had a maximum transverse width W32 (e.g., diameter), but has broken into components, which include a first small aggregate 346A, a second small aggregate 346B, and individual magnetic particles 340. All of the components are smaller than the width W32 and are small enough to pass through the mesh 312 and the inlet valve 214A (FIG. 2B) of the pump 106.

A second large aggregate 344 may have a maximum transverse width W33 (FIG. 3A) that is greater than the widths W31 of the spaces 334. The second large aggregate 344 is shown approaching a space 334C. As the second large aggregate 344 attempts to pass through the space 334C, the second large aggregate 344 collides with the members 332 forming the space 334C. The collision disassociates the magnetic particles 340 of the second large aggregate 344 and causes the second large aggregate 344 to break into smaller components. In the embodiment of FIG. 3C, the second large aggregate 344 has broken into three small aggregates shown as a third small aggregate 346C, a fourth small aggregate 346D, and a fifth small aggregate 346E, and some magnetic particles 340. The components have widths smaller than the widths W31 of the mesh 312 and the transverse inlet dimension D21 (FIG. 2B) of the inlet valve 214A, so the components will not clog and/or damage the inlet valve 214A.

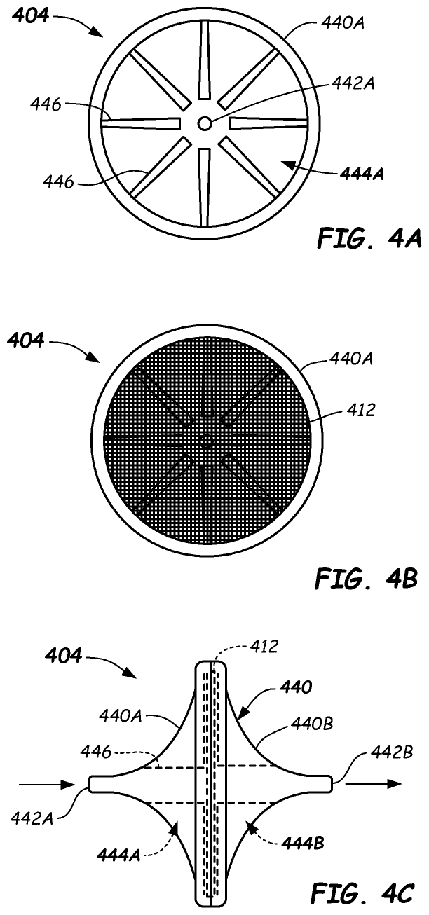

Reference is now made to FIGS. 4A-4C, which illustrate various components of an example of a mesh apparatus 404. FIG. 4A illustrates a plan view of a housing first portion 440A without a mesh 412 located therein. FIG. 4B illustrates a plan view of the housing first portion 440A with the mesh 412 located therein. FIG. 4C illustrates a side elevation view of a mesh apparatus 404.

Referring to FIG. 4A, the housing first portion 440A is shown as having a circular outer perimeter. The housing first portion 440A may be other shapes, such as square or oval. The housing first portion 440A may include a housing inlet 442A that receives a liquid, such as from the liquid/particle source 102 (FIGS. 1A-1B). The housing first portion 440A may have a first cavity 444A that receives and/or contains the liquid and particles/aggregates. As shown in FIG. 4C, the housing first portion 440A may be affixed to a housing second portion 440B to form a housing 440. The housing second portion 440B may have a housing outlet 442B (FIG. 4C) that discharges the liquid and magnetic particles 340/small aggregates 346, such as to the pump 106 (FIGS. 1A-1B). The housing second portion 440B may include a second cavity 444B that contains the liquid and particles 340/small aggregates 346. In some embodiments, the housing second portion 440B may be identical or substantially similar to the housing first portion 440A.

The housing first portion 440A may include one or more supports 446 that retain the mesh 412 in a fixed location within the housing 440. The mesh 412 may have spaces and members (e.g., wires) identical or similar to the mesh 312 (FIGS. 3A-3C). The mesh 412 may be cut or formed to an appropriate size and set on the supports and within the housing first portion 440A. The housing second portion 440B may then be affixed to the housing first portion 440A as shown in FIG. 3C to form the housing 440, such as by adhesive or mechanical connection. The location of the mesh 412 within the housing 440 may provide for all liquid passing between the housing inlet 442A and the housing outlet 442B to pass through the mesh 412. Accordingly, aggregates of magnetic particles will be disassociated into small aggregates 346 (FIG. 3C) and or individual magnetic particles 340 as described with reference to FIGS. 3A-3B.

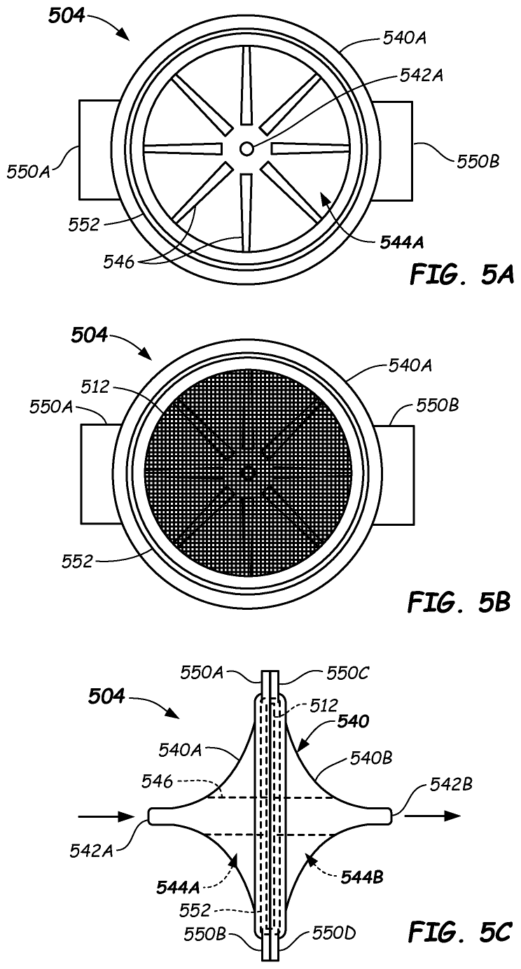

Reference is now made to FIGS. 5A-5C, which illustrate various components of another embodiment of a mesh apparatus 504. FIG. 5A illustrates a plan view of a housing first portion 540A without a mesh 512 located therein. FIG. 5B illustrates a plan view of the housing first portion 540A with the mesh 512 located therein. FIG. 5C illustrates a side elevation view of the mesh apparatus 504.

Referring to FIG. 5A, the housing first portion 540A may be similar to the housing first portion 440A (FIG. 4A). The housing first portion 540A is shown as being round, but may be other shapes, such as square or oval. The housing first portion 540A may include a housing inlet 542A that receives a liquid and particles/aggregates, such as from the liquid/particle source 102 (FIGS. 1A-1B). The housing first portion 540A may have a first cavity 544A that receives and/or contains the liquid and particles/aggregates. As shown in FIG. 5C, the housing first portion 540A may be affixed to a housing second portion 540B by the use of tabs 550A-550D to form a housing 540. The housing second portion 540B may include a housing outlet 542B that discharges the liquid and magnetic particles 340/small aggregates 346 (FIG. 3C), such as to the pump 106 (FIGS. 1A-1B). The housing second portion 540B may include a second cavity 544B that contains the liquid. In some embodiments, the housing second portion 540B may be identical or substantially similar to the housing first portion 540A.

The housing first portion 540A may include one or more supports 546 that retain the mesh 512 in a fixed location within the housing 540. The mesh 512 may have spaces and members (e.g., wires) identical or similar to the mesh 312 (FIGS. 3A-3C). The mesh 512 may be cut or formed to an appropriate size and set on the supports 546 within the housing first portion 540A. The housing first portion 540A may include a first tab 550A and a second tab 550B that extend from the exterior of the housing first portion 540A. The housing second portion 540B may include a third tab 550C and a fourth tab 550D that extend from the exterior of the housing second portion 540B. The first tab 550A may engage the third tab 550C and the second tab 550B may engage the fourth tab 550D to couple the housing first portion 540A to the housing second portion 540B to form the housing 540. In some embodiments, screws or other fasteners may be placed through the first tab 550A and into the third tab 550C and through the second tab 550B and into the fourth tab 550D to attach the housing first portion 540A to the housing second portion 540B.

The housing first portion 540A may include a gasket 552 that may be located in a groove or the like (not shown). The gasket 552 may be located outside of the mesh 512 and may prevent liquids from exiting a joint between the housing first portion 540A and the housing second portion 540B.

Additional reference is made to the embodiment of the mesh apparatus 104 of FIG. 1B. The mesh apparatus 104 includes a mesh 112 that may be substantially similar to the mesh 312 (FIG. 3A), the mesh 412 (FIG. 4B), or the mesh 512 (FIG. 5B). The container 110 may have a first cavity 144A located on an inlet side of the mesh 112 and a second cavity 144B located on an outlet side of the mesh 112. The mesh apparatus 104 may store the liquid received from the liquid/particle source 102 until the pump 106 has time to pump the liquid from the mesh apparatus 104. In some embodiments, the liquid may only be stored in the mesh apparatus 104 for periods that are short so that the magnetic particles do not have time to attract to each other and form new aggregates. The term housing, as used herein, can have any suitable structure adapted to contain and support the mesh, and can be integrated into a conduit or a pump, for example.

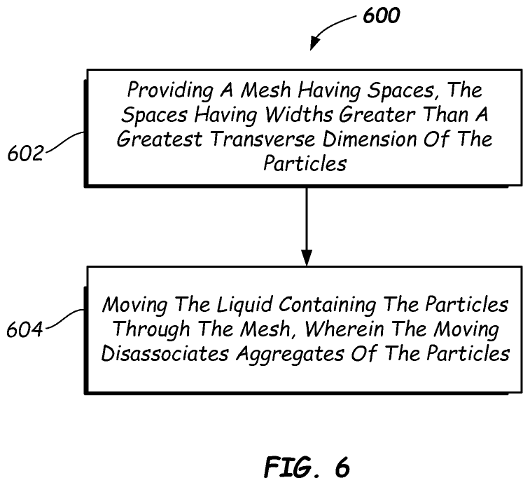

In another aspect, a method of transferring a liquid containing particles (e.g., magnetic particles 340) is disclosed and described in the flowchart of FIG. 6. The method 600 includes, in 602, providing a mesh (e.g., mesh 312) having spaces (e.g., spaces 334), the spaces having widths (e.g., widths W31) greater than a greatest transverse dimension of the particles. The method includes, in 604, moving the liquid containing the particles through the mesh, wherein the moving disassociates aggregates (e.g., large aggregates 342, 344) of the particles.

The liquid transfer system 100 and embodiments thereof have been described herein as transporting liquids including magnetic particles. The liquid transfer system 100 and embodiments thereof may transport liquids including other particles. For example, the liquid transfer system 100 and embodiments thereof may transport liquids containing particles that may form aggregates by adhesion or other forces. For example, the particles may have protein coatings wherein the protein coatings attract the particles together so as to form aggregates.

While the disclosure is susceptible to various modifications and alternative forms, specific assembly and apparatus embodiments and methods thereof have been shown by way of example in the drawings and are described in detail herein. It should be understood, however, that it is not intended to limit the disclosure to the particular assemblies, apparatus, or methods disclosed, but, to the contrary, the intention is to cover all modifications, equivalents, and alternatives falling within the scope of the claims.

What is claimed is:

1. An apparatus configured to receive particles in a liquid, comprising:
a housing comprising a housing inlet and a housing outlet; and
a mesh located in the housing between the housing inlet and the housing outlet, the mesh having spaces greater than a greatest transverse dimension of the particles.

2. The apparatus of claim 1, wherein the spaces are square.

3. The apparatus of claim 1, wherein the spaces have widths less than 1.3 mm.

4. The apparatus of claim 1, wherein the spaces have widths in a range from 1.15 mm to 1.25 mm.

5. The apparatus of claim 1, wherein the housing is made of nonmagnetic material.

6. The apparatus of claim 1, wherein the apparatus is configured to be coupled to a pump having an inlet valve, wherein the inlet valve having a transverse inlet dimension, and wherein the spaces are smaller than the transverse inlet dimension of the inlet valve.

7. The apparatus of claim 1, wherein the mesh is made of a nonmagnetic material.

8. The apparatus of claim 1, wherein the particles are magnetic particles.

9. The apparatus of claim 1, wherein the mesh includes members located between the spaces and wherein the members have thicknesses in a range from 0.127 mm to 0.381 mm.

10. The apparatus of claim 1, wherein the mesh has an open area in a range from 31% to 41%.

11. The apparatus of claim 1, comprising first wires extending in a first direction and second wires extending in a second direction, wherein the first wires are woven together with the second wires, and wherein the spaces are located between first wires and the second wires.

12. A clinical diagnostic analyzer, comprising:
a pump configured to pump a liquid containing particles;
a mesh apparatus configured to disassociate aggregates of the particles, the mesh apparatus comprising:
a housing comprising a housing inlet and a housing outlet coupled to the pump; and
a mesh located in the housing between the housing inlet and the housing outlet, the mesh having spaces greater than a greatest transverse dimension of the particles.

13. The clinical diagnostic analyzer of claim 12, wherein the clinical diagnostic analyzer is implemented in an immunoassay instrument.

14. The clinical diagnostic analyzer of claim 12, wherein the particles are magnetic particles.

15. The clinical diagnostic analyzer of claim 12, wherein the spaces have widths in a range from 1.15 mm to 1.25 mm.

16. The clinical diagnostic analyzer of claim 12, wherein the spaces have widths less than 1.3 mm.

17. The clinical diagnostic analyzer of claim 12, wherein the spaces have widths in a range from 1.15 mm to 1.25 mm.

18. The clinical diagnostic analyzer of claim 12, wherein the mesh comprises first wires extending in a first direction and second wires extending in a second direction, wherein the first wires are woven together with the second wires, and wherein the spaces are located between first wires and the second wires.

19. The clinical diagnostic analyzer of claim 12, wherein the mesh includes one or more members located between the spaces and wherein the members have thicknesses in a range from 0.127 mm to 0.381 mm.

20. The clinical diagnostic analyzer of claim 12, wherein the mesh has an open area in a range from 31% to 41%.

21. A method of transferring a liquid containing particles, comprising:
providing a mesh having spaces, the spaces having widths greater than a greatest transverse dimension the particles; and
moving the liquid containing the particles through the mesh, wherein the moving disassociates aggregates of the particles.

22. The method of claim 21, wherein the particles are magnetic particles.
